(19) United States
(12) Patent Application Publication
(10) Pub. No.: US 2013/0204113 A1
(43) Pub. Date: Aug. 8, 2013

(54) MULTI-MODALITY COMPACT BORE IMAGING SYSTEM

(71) Applicant: Raz Carmi, Haifa (IL)

(72) Inventor: Raz Carmi, Haifa (IL)

(73) Assignee: KONINKLIJKE PHILIPS ELECTRONICS N.V., EINDHOVEN (NL)

(21) Appl. No.: 13/876,172

(22) PCT Filed: Oct. 5, 2011
(86) PCT No.: PCT/IB2011/054383

§ 371 Date: Mar. 27, 2013

Related U.S. Application Data
(60) Provisional application No. 61/392,608, filed on Oct. 13, 2010.

(51) Int. Cl. A61B 6/00; A61B 7/00; A61B 5/04; A61B 5/00; A61B 6/03

(52) U.S. Cl. CPC A61B 6/4417; A61B 5/0035; A61B 6/037; A61B 6/032; A61B 5/0075; A61B 5/04008; A61B 5/0042; A61B 6/501; A61B 5/4088; A61B 5/4082; A61B 5/4094; A61B 5/0077; A61B 7/00
USPC 600/407

(57) ABSTRACT

A multi-modality imaging system (100) includes a gantry (101), including at least first and second imaging modalities (102, 104) respectively having first and second bores (113, 112) arranged with respect to each other along a z-axis, and a subject support (108) that supports a subject for scanning. The gantry is configured to alternately move to a first position at which the subject support extends into the first bore of first imaging modality for scanning an extremity of the subject and to a second position at which the subject support extends into the second bore of second imaging modality for scanning the extremity of the subject.

The following generally relates to multi-modality imaging and finds particular application to a multi-modality compact bore imaging system such as positron emission tomography—x-ray computed tomography (PET/CT), single photon emission computed tomography—x-ray computed tomography (SPECT/CT), positron emission tomography—magnetic resonance imaging (PET/MRI), and/or other multi-modality imaging systems, including imaging systems such as infrared imaging, magnetic-particle imaging (MPI), magneto-encephalography (MEG), and other medical and non-medical imaging systems.

Dual-modality imaging systems include PET/CT, SPECT/CT, and PET/MRI systems. Usually, one of the modalities is used to image functional information (e.g., PET or SPECT) and the second modality is used to image anatomical information (e.g., CT or MRI). Generally, the anatomical modality provides important anatomical information, relatively better localization of the functional data through geometrical registration and fusion visualization. In PET and SPECT, the anatomical image improves the functional image quality and provides better quantitative diagnostics by applying radiation attenuation correction. Attenuation correction with MRI is still problematic but sufficient solutions may exist at least for brain imaging. Additional dual-modality approaches such as synergistic enhancement of the functional images improves the PET or SPECT spatial resolution, enhances image contrast, corrects partial-volume effects, reduces image-noise, and can add to the functional images fine structures, which may appear in the anatomical images.

The information provided by dual-modality imaging systems can provide accurate quantitative diagnosis, high spatial resolution, and artifact free images. With particular relevance to compact bore systems suited for brain imaging, the foregoing, generally, can be important in diagnosis and/or early detection, for example, with diseases such as Alzheimer, Parkinson, Epilepsy, Autism, prion-related, Stroke, Cancer, or other diseases where detection and treatment, usually before symptoms appear, may slow or even halt the disease progression. Unfortunately, conventional dual-modality imaging systems have been formed by integrating large bore commercial full body imaging systems. As a consequence, conventional dual-modality imaging systems tend to be costly, may provide less than desired performance for small object imaging, and are not well-suited for certain procedures or studies since these systems generally are optimized for larger objects such as the human shoulders, pelvis, or the torso, or the entire body.

Aspects of the present application address the above-referenced matters and others.

According to one aspect, a multi-modality imaging system includes a gantry, including at least first and second imaging modalities respectively having first and second bores arranged with respect to each other along a z-axis, and a subject support that supports a subject for scanning The gantry is configured to alternately move to a first position at which the subject support extends into the first bore of first imaging modality for scanning an extremity of the subject and to a second position at which the subject support extends into the second bore of second imaging modality for scanning the extremity of the subject.

In another aspect, a method includes loading a sub-portion of a subject, via a subject support, into a first bore of a first imaging modality of a gantry of a multi-modality imaging system along a z-axis, performing a first scan of the sub-portion utilizing the first imaging modality, and unloading the sub-portion from the first bore. The method further includes rotating the gantry to position a second imaging modality of the multi-modality imaging system for imaging the sub-portion. The method further includes loading the sub-portion, via the subject support, into a second bore of the second imaging modality of the gantry along the z-axis, performing a second scan of the sub-portion utilizing the second imaging modality, and unloading the sub-portion from the second bore.

In another aspect, an imaging system includes a subject support that translates between a first position in which a subject to be scanned is outside of an imaging region and a second position in which the subject is in the imaging region, and two or more imaging modalities that are selectively movable to be positioned at the imaging region.

Still further aspects of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

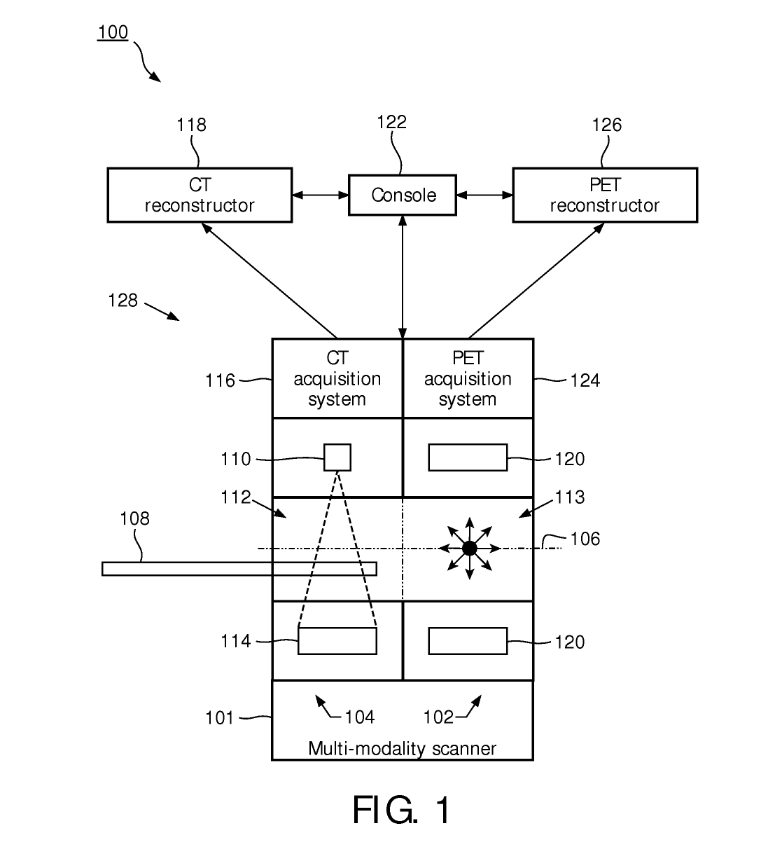

FIG. 1 illustrates an example multi-modality imaging system.

FIGS. 2, 3, 4 and 5 illustrate an example multi-modality imaging system configured to rotate on a base between imaging modalities.

FIG. 6 illustrates an example multi-modality imaging system configured to rotate in space between imaging modalities.

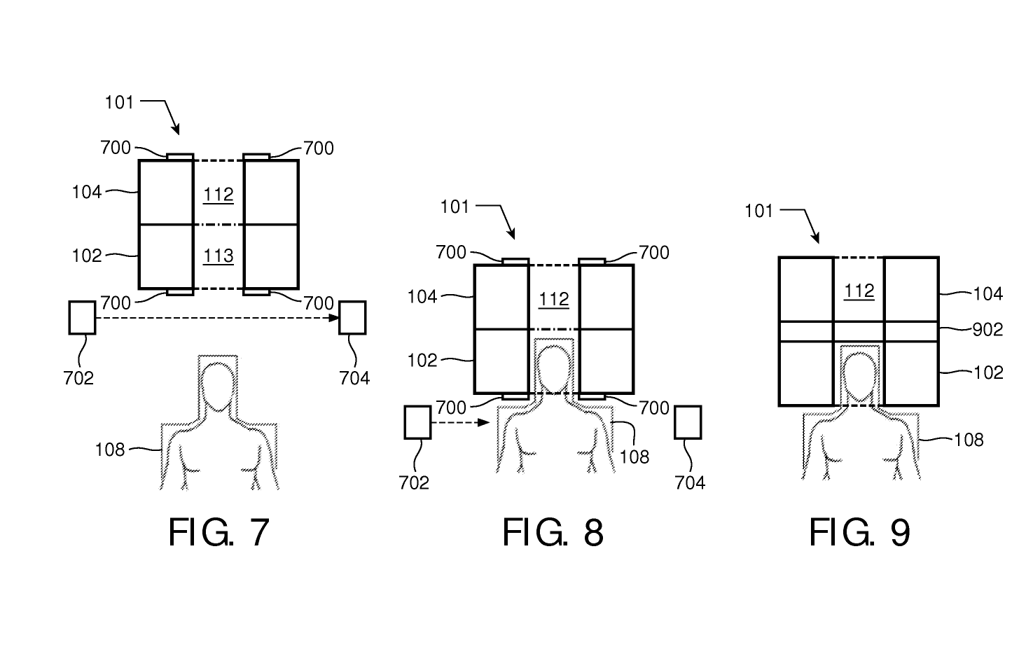

FIGS. 7 and 8 illustrate an example collision detection sensor in connection with the multi-modality imaging system.

FIG. 9 illustrates an example multi-modality imaging system in which a device is disposed between the imaging modalities.

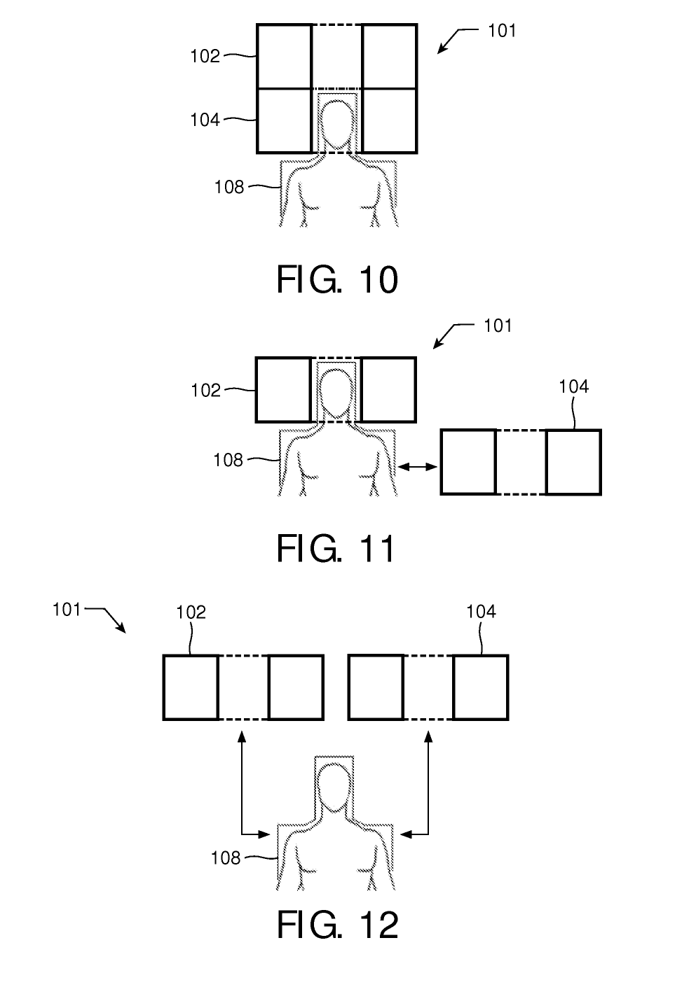

FIGS. 10 and 11 illustrate an example in which at least one of the imaging modalities slides into and out of position for scanning

FIG. 12 illustrates an example in which the imaging modalities are aligned side by side and the patient support moves between the modalities.

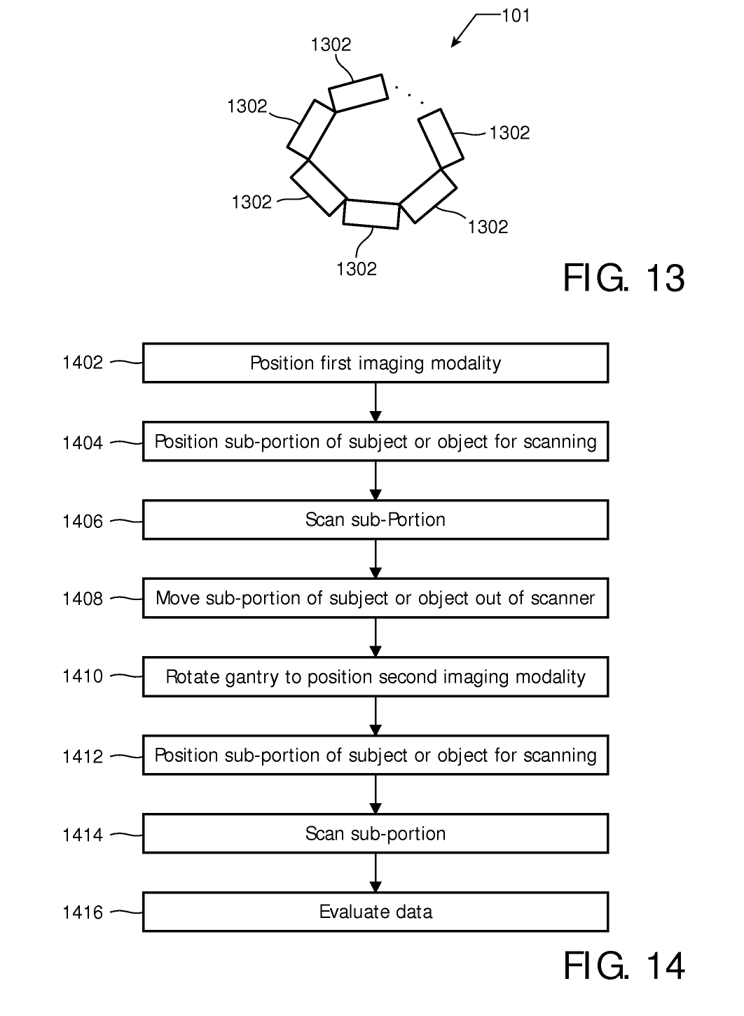

FIG. 13 illustrates an example with a plurality of imaging modalities.

FIG. 14 illustrates a method.

FIG. 1 illustrates a multi-modality imaging system 100, which includes a combined positron emission tomography/x-ray computed tomography (PET/CT) gantry 101 with both a PET gantry portion 102 and a CT gantry portion 104. In another embodiment, the CT gantry portion 104 is replaced with another imaging modality such as a magnetic resonance (MR) gantry portion. Additionally or alternatively, the PET gantry portion 102 is replaced with another imaging modality such as a single photon emission computed tomography (SPECT) gantry portion. Other combinations are also contemplated herein. Furthermore, such combinations may include three or more imaging systems.

The CT portion 104 includes a radiation source 110 such as an x-ray tube that rotates around a bore 112, which defines a CT examination region, about a z-axis 106.

An x-ray radiation sensitive detector array 114 detects radiation that traverses the examination region 112 and generates a signal indicative thereof. A CT acquisition system 116 processes the signal and generates CT projection data indicative of the detected radiation. A CT reconstructor 118 reconstructs the CT projection data and generates volumetric image data indicative of the examination region and any structure disposed therein.

The PET gantry portion 102 includes a gamma ray radiation sensitive detector array 120 disposed about a bore 113, which defines a PET examination region.

The detector 120, in response to receiving a gamma ray characteristic of a positron annihilation event occurring in the examination region, generates a signal indicative thereof. A PET data acquisition system 124 processes the signal and generates PET projection data such as a list of detected annihilation events, a time at which an event was detected, and position and orientation of the corresponding line-of-response (LOR). Where the portion 102 is configured with time-of-flight (TOF) capabilities, an estimate of the position of the annihilation along the LOR is also provided. A PET reconstructor 126 reconstructs the PET projection data and generates image data indicative of the distribution of the radionuclide in a scanned object or subject.

In the illustrated embodiment, the multi-modality scanner 100 is configured as a compact multi-modality scanner in which the bores 112 and 113 respectively have a physical dimension that corresponds to a predetermined object size. For example, in one embodiment, at least one of the bores 112 and 113 has a physical dimension that corresponds to a size of a human head, arm, leg, or other extremity. With this embodiment, generally, the bores 112 and 113 are not large enough to receive the shoulders, torso, pelvis, and/or other regions of the body. In this embodiment, the bores 112 and 113 may have a same or different size. Such a scanner can be dedicated to and/or optimized for a particular object and/or object size.

In another embodiment, at least one of the bores 112 and 113 has a physical dimension that corresponds to an animal (e.g., a mouse, a dog, etc.) head, leg, tail, or other extremity. Likewise, generally, the bores 112 and 113 may not be large enough to receive the entire and/or other portions of the body of certain animals. In yet another embodiment, at least one of the bores 112 and 113 has a physical dimension that corresponds to a sub-portion of an object, for example, for non-destructive testing, luggage inspection, etc. Similarly, the bores 112 and 113 will generally may be large enough to receive the entire object and/or other portions of the object.

By having such bores 112 and 113, the system 100 may be relatively compact, low in cost, have a small footprint, and be low weight (which may allow for mobility), for example, relative to a configuration supporting whole body scanning In addition, the small geometrical configuration of the bores 112 and 113 enables improved imaging optimization for smaller objects, such as higher spatial resolution in PET, and better relation between image-quality to radiation dose in CT. As described in greater detail below, the multi-modality scanner 100 is configured to be moveable so that a particular one of the modalities 102 or 104 can be positioned for scanning the sub-portion of the subject or object.

In the illustrated embodiment, the PET gantry portion 102 and the CT gantry portion 104 are disposed back to back along a common longitudinal or z-axis 106. A support 108 supports an object or subject for imaging the sub-portion of the object or subject in an examination region 112. In the illustrated embodiment, the support 108 loads and unloads a sub-portion of the object or subject from only one (loading) side 128 of the system 100. In this embodiment, the support 108 physically translates into only the gantry portion 102 or 104 facing the loading side 128 and cannot translate, in case where the examined subject is loaded, through the bore to the other gantry portion. As described in greater detail below, and in order to switch between the gantry portions 102 and 104, the support 108 is moved sufficiently away from loading side 128, and the gantry 101 is moved so that the other gantry portion 102 or 104 faces the loading side 128.

An operator console 122 such as a computer includes a human readable output device such as a monitor or display and input devices such as a keyboard and mouse. A processor of the console 122 executes software or computer readable instructions encoded on computer readable storage medium, which allows the operator to perform functions such as selecting a dual imaging protocol, moving the patient support in and out of the bores 112 and 113, initiating scanning, viewing and/or manipulating the acquired data (e.g., fusing dual modality data), etc.

As briefly discussed above, in one embodiment the combined modality gantry 101 is moveable, which allows the combined modality gantry 101 to be moved at least between a position at which the CT gantry portion 102 can be used to image the portion of the subject or object on the subject support 108 and at which the PET gantry portion 104 can be used to image the portion of the subject or object on the subject support 108. FIGS. 2, 3, 4 and 5 illustrate a non-limiting example of such a gantry 101.

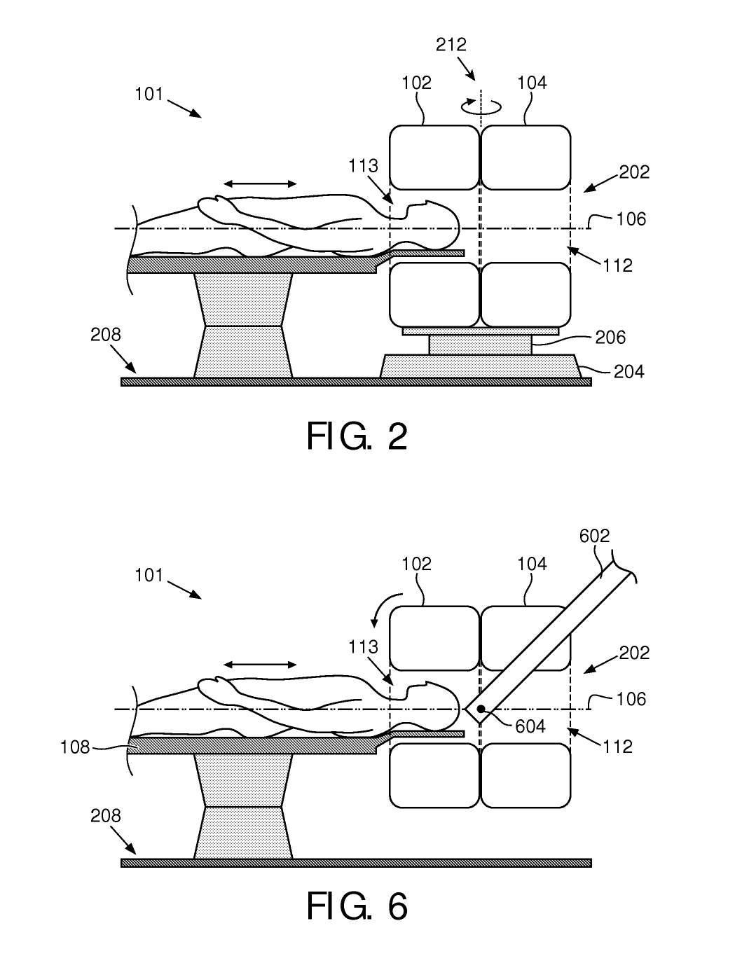

Initially referring to FIGS. 2 and 3, the bores 112 and 113 have a physical dimension 202 corresponding to a head of a human patient. In other words, in this embodiment, a geometry or size (e.g., the volume) of the bores 112 and 113 is such that an object the size of a human head (e.g., average size plus a margin) or smaller will fit in the bores 112 and 113, but an object that is larger the human head will not fit in the bores 112 and 113.

With continuing reference to FIG. 2, the gantry 101 is affixed, through a coupling 206, to a member or base 204 which is mounted to or rests on a surface. With this embodiment, the coupling 206 rotatably couples the gantry 101 to the base 204. Electrical power leads to the two portions 102 and 104 may be designed using a technology such as “electrical brushes on rails,” “slip ring” or similar, as an alternative to using somewhat inconvenient flexible moving power cables.

Various approaches can be used to rotate the gantry 101 with respect to the base 204. For example, in one instance the system 100 includes a motor, a drive (e.g., belt, gears, etc.), and a controller, which receives a command signal from the console 122 and controls the drive to control the motor to rotate the gantry 101. In the illustrated embodiment, the gantry 101 rotates about an axis 212 which is substantially perpendicular to both the axis 106 and to the surface 208 which supports the base 204 and the subject support 108. In another embodiment, the gantry 101 is configured so that a user can manually rotate the gantry 101.

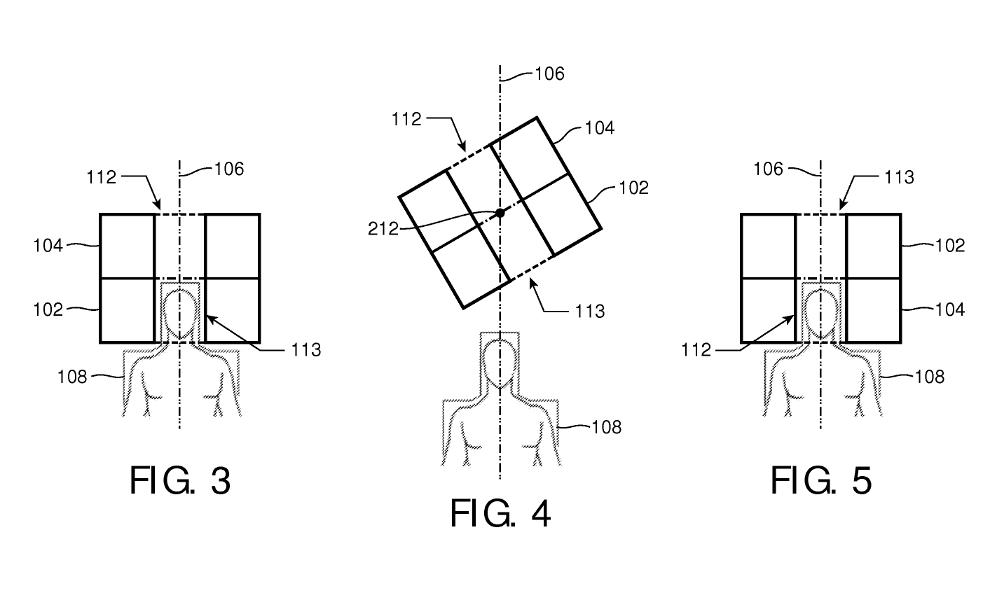

FIGS. 3, 4 and 5 show an example of switching between the portions 102 and 104. In FIG. 3, the gantry 101 is positioned so that the imaging portion 102 faces the patient support 108, and the patient support 108 is in an extended position in which the head of the patient is in the bore 113 of the imaging portion 102. In FIG. 4, the patient support 108 is in a retracted position in which the head of the patient is outside of the bore 113, and the gantry 101 is rotating about to the axis 212, pointing out of the plane of FIG. 4.

In FIG. 5, the gantry 101 is positioned so that the imaging portion 104 faces the patient support 108, and the patient support 108 is in an extended position in which the head of the patient is in the bore 112 of the imaging portion 104. The system 100 may be configured to ensure accurate geometrical image registration between the two imaging portions 102 and 104. In one instance, this may include affixing special instruments for calibrating the geometrical registration between the two modalities, and to ensure the accuracy of system positioning after rotation.

With further respect to FIGS. 3, 4 and 5, in one embodiment, the gantry 101 is configured to rotate about the axis 212 in one direction one or more revolutions to switch between the imaging portions 102 and 104 back and forth. In another embodiment, the gantry 101 is configured to rotate one hundred and eighty degrees (180°) in one direction to switch between the imaging portions 102 and 104, and then 180° in the other direction to switch between the imaging portions 104 and 102.

With further respect to FIGS. 3, 4 and 5, in another embodiment, the gantry 101 rotates about an axis substantially perpendicular to the axis 106 and parallel to the surface 208. An example of this is shown in FIG. 6, in which the gantry 101 is carried by a member or support 602 and rotates about an axis 604, which is perpendicular to the axis 106 and parallel to the plane 208. In yet another embodiment, the gantry 101 rotates in connection with both axes 212 and 604, in series or in parallel. In a variation of this embodiment, the gantry 101 is tilted relative to the floor (and not vertical to the floor plane). In this instance, the patient is placed at a suitable incline with respect to the floor so that the patient can be scanned by the system 100.

As shown in FIGS. 7 and 8, the system 100 may include one or more collision sensors. By way of example, in the illustrated embodiment, pressure sensors 700 are positioned on the gantry 101 adjacent to the opening into the bores 112 and 113, and sense contact, for example, by the patient support 108 or other object that physically contacts the pressure sensors 700. The pressure sensors 700 generate a signal indicative of such contact, and the signal is conveyed to the console 122, which triggers invocation of a collision routine that stops and/or reverses patient support 108 and/or otherwise mitigates a collision.

Other suitable sensors include, but are not limited to, an optical, radio frequency, infrared, magnetic, acoustic, and/or other proximity sensor and/or other sensor that acquires information that can be used for collision monitoring such as a camera, video recorder, and/or the like. Furthermore, such sensors may be located on one or more of the sides of the gantry 101 for collision monitoring with objects (e.g., IV poles, EKG instruments, radiation shields, etc.) next to gantry 101 and/or personnel that might be in the exam room.

In another example, a light source 702 emits a light beam, and a detector 704 is configured to detect the light beam. As shown in FIG. 7, when the patient support 108 is outside of the light beam, the light beam is detected by the detector 704, which generates a signal indicative thereof. The signal can be conveyed to console 122 and used as a trigger for allowing the gantry 101 to move between gantry 101 positions. As shown in FIG. 8, when the patient support 108 or other object inhibits the light beam from reaching the detector 704, then the gantry 101 is inhibited from rotating.

It is to be appreciated that one, both or neither of the above collision may be used with the system 100. Furthermore, one or more other collision devices may additionally or alternatively be used with the system 100.

FIG. 9 illustrates an embodiment in which a device 902 is disposed between the imaging modalities 102 and 104. Where one of the modalities includes an MRI imaging system, the device 902 may include a magnetic shield, a cooler, power supply, computers, etc., which can be affixed to the gantry 101. Additionally or alternatively, where one of the modalities includes a CT, SPECT or PET imaging system, the device 902 may include bearings respectively for rotating an x-ray source and an x-ray detector or a gamma ray detector.

FIGS. 10 and 11 illustrate an embodiment in which the modality 104 translates to a first position in front of the modality 102 for scanning the subject on the subject support 108 with the modality 104 and to a second position in which the modality 102 can be used for scanning the subject on the subject support 108. In another alternative embodiment, the location of the portions with respect to the subject support 108 is reversed, and the modality 102 translates to the first position in front of the modality 104 for scanning the subject on the subject support 108 with the modality 102 and to the second position in which the modality 104 can be used for scanning the subject on the subject support 108.

FIG. 12 illustrates an embodiment in which the modalities 102 and 104 are placed side-by-side at fixed locations, and the patient support 108 moves between the two modalities 102 and 104 to enable scanning with each one of the modalities. With this configuration, the patient support 108 may move on rails or freely on wheels.

FIG. 13 illustrates an embodiment in which the gantry 101 includes N modalities 1302, wherein N is an integer equal to or greater than two. In this embodiment, the N modalities 1302 are arrangement in a circular arrangement. In other embodiments, other arrangements may also used.

Other mechanical couplings and/or methods of positioning of the modalities are also contemplated.

FIG. 14 illustrates a method.

At 1402, a multi-modality imaging system is positioned so that a first imaging modality of the multi-modality imaging system faces a subject support supporting a subject or object to be scanned.

At 1404, a sub-portion of the subject or object is positioned in an examination region of the first imaging modality. As described herein, the examination region is defined by a size of a bore of the system, which corresponds to a size of a particular object scanned by the system.

At 1406, the sub-portion is scanned.

At 1408, the subject or object is moved out of the examination region.

At 1410, the multi-modality imaging system is rotated so that a second imaging modality of the multi-modality imaging system faces the subject support supporting a subject or object.

At 1412, a sub-portion of the subject or object is positioned in an examination region of the second imaging modality.

At 1414, the sub-portion is scanned.

At 1416, the data from one or more of the scans can be evaluated. For example, in one instance the data can be used for assessing brain functionality, physiology, anatomy or other conditions, including the usage of special tracers, contrast materials, or agents. Possible clinical applications can be early detection and follow-up of Alzheimer's disease, imaging of brain tumors, assessing neurological functionality and more, such as Parkinson, Epilepsy, Autism, prion-related, Stroke, Cancer, etc.

Those of ordinary skill in the art will recognize that the various techniques described above may be implemented by way of computer readable instructions stored on a computer readable storage medium accessible to a computer processor. Additionally or alternatively, the readable instructions can be stored on signal or other transitory medium. When executed, the instructions cause the processor(s) to carry out the described techniques.

The invention has been described with reference to various embodiments. Modifications and alterations may occur to others upon reading the detailed description. It is intended that the invention be constructed as including all such modifications and alterations insofar as they come within the scope of the appended claims or the equivalents thereof.

1. A multi-modality imaging system, comprising:
a gantry, including at least first and second imaging modalities respectively having first and second bores arranged with respect to each other along a z-axis; and
a subject support that supports a subject for scanning,
wherein the gantry is configured to alternately move to a first position at which the subject support extends into the first bore of first imaging modality for scanning an extremity of the subject and to a second position at which the subject support extends into the second bore of second imaging modality for scanning the extremity of the subject.

2. The system of claim I, wherein the first and second bores have physical dimensions corresponding to a physical dimension of the extremity,

3. The system of claim 2, wherein the first and second bores have physical dimensions that are smaller than the physical dimension of a sub-portion of the subject with a physical dimension that is larger than the physical dimension of the extremity.

4. The system of claim 1, wherein the subject support, when loaded with the subject, is configured to extend into only one of the bores at each of the positions.

5. The system of claim 1, further comprising:
a base; and
a coupling that moveably couples the gantry to the base,
wherein the gantry is configured to move via the coupling about the base to move between the first and second positions.

6. The system of claim 5, wherein the gantry rotates about an axis perpendicular to the z-axis of the gantry.

7. The system of claim 1, wherein the first and second positions are spatially registered with respect to each other,

8. The system of claim 1, wherein the first and second modalities are aligned with respect to each other back to back.

9. The system of claim 8, further comprising at least one of a shield or a bearing of one of the modalities between the first and second modalities.

10. The system of claim 1, further comprising a collision sensor that allows or prevents movement of the gantry, based on a location of the subject or the subject support with respect to the gantry.

11. The system of claim 1, wherein the system is a dedicated head scanner, the first and second bores have a physical dimension for optimizing head scans, and the extremity is a head of the subject.

12. A method, comprising:
loading a sub-portion of a subject, via a subject support, into a first bore of a first imaging modality of a gantry of a multi-modality imaging system along a z-axis;
performing a first scan of the sub-portion utilizing the first imaging modality;
unloading the stab-portion from the first bore;
rotating the gantry to position a second imaging modality of the multi-modality imaging system for imaging the sub-portion;
loading the sub-portion, via the subject support, into a second bore of the second imaging modality of the gantry along the z-axis;
performing a second scan of the stab-portion utilizing the second imaging modality; and
unloading the stab-portion from the second bore.

13. The method of claim 12, further comprising:
reconstructing the data from at least one of the first or second scans; and
generating one or more images from the reconstructed data.

14. The method of claim 12, wherein the first and second bores have physical dimensions that correspond to a physical dimension of the sub-portion.

15. The method of claim 12, further comprising:
rotating the gantry about one hundred and eighty degrees to position the second imaging modality for imaging the sub-portion.

16. The method of claim 15, further comprising:
rotating the gantry about an axis perpendicular to both the z-axis of the gantry and the surface supporting the subject support.

17. The method of claim 15, further comprising:
rotating the gantry about an axis perpendicular to the z-axis of the gantry and parallel to the surface supporting the subject support.

18. The method of claim 12, further comprising:
rotating the gantry to position at least a third imaging modality of the multi-modality imaging system for imaging the sub-portion;
loading the sub-portion, via the subject support, into at least a third bore of the third imaging modality of the gantry along the z-axis;
performing a third scan of the sub-portion utilizing the third imaging modality; and
unloading the sub-portion from the second bore.

19. An imaging system, comprising:
a subject support that translates between a first position in which a subject to be scanned is outside of an imaging region and a second position in which the subject is in the imaging region; and
two or more imaging modalities that are selectively movable to be positioned at the imaging region.

20. The system of claim 19, wherein the modalities are on a common gantry.

21. The system of claim 19, wherein the modalities are coupled to provide alignment between modalities.
